(19) United States
(12) Patent Application Publication
(10) Pub. No.: US 2010/0166698 A1
(43) Pub. Date: Jul. 1, 2010

(54) NEW COMPOSITIONS AND METHODS FOR THE TREATMENT OF INFLAMMATION

(75) Inventor: Jayson M. Rieger, Charlottesville, VA (US)

Correspondence Address: SCHWEGMAN, LUNDBERG & WOESSNER, P.A., P.O. BOX 2938, MINNEAPOLIS, MN, 55402, US

(73) Assignee: University of Virginia Patent Foundation

(21) Appl. No.: 12/630,231

(22) Filed: Dec. 3, 2009

Related U.S. Application Data
(63) Continuation of application No. 11/497,280, filed on Aug. 2, 2006, now abandoned.
(60) Provisional application No. 60/704,433, filed on Aug. 2, 2005.

(51) Int. Cl. A61K 31/7076; A61K 39/395; A61K 38/17; A61K 38/48; A61K 38/19; A61K 38/08; A61P 29/00

(52) U.S. Cl. 424/85.1; 514/46; 424/141.1; 514/12; 424/94.63; 424/134.1; 424/133.1; 424/145.1; 514/11

(57) ABSTRACT

The present invention describes combinations of A_{2A} adenosine receptor agonists and anti-inflammatory compounds for the inhibition of an inflammatory response in mammalian tissue.

This application is a continuation of U.S. patent application entitled: “New compositions and methods for the treatment of inflammation”, filed Aug. 2, 2006, Ser. No. 11/497,280, which claims priority to U.S. Provisional Patent Application Ser. No. 60/704,433 filed Aug. 2, 2005, entitled “Compositions and Methods for the Treatment of Inflammation”, the contents of which are incorporated herein by reference in their entirety.

### BACKGROUND OF THE INVENTION

There is a wide range of pathogenic insults that can initiate an inflammatory response including infection, allergens, autoimmune stimuli, immune response to transplanted tissue, noxious chemicals, and toxins, ischemia/reperfusion, hypoxia, mechanical and thermal trauma. Inflammation typically is a localized action which serves in expulsion, attenuation by dilution, and isolation of the damaging agent and injured tissue. The body's response becomes an agent of disease when it results in inappropriate injury to host tissues in the process of eliminating the targeted agent, or responding to a traumatic insult.

Inflammation is a component of pathogenesis in several vascular diseases or injuries. Examples include: ischemia/reperfusion injury (N. G. Frangogiannis et al., in Myocardial Ischemia: Mechanisms, Reperfusion, Protection, M. Karmazyn, ed., Birkhuser Verlag (1996) at 236-284; H. S. Sharma et al., Med. of Inflamm., 6, 175 (1987)), atherosclerosis (R. Ross, Nature, 362, 801 (1993)), inflammatory aortic aneurysms (N. Girardi et al., Ann. Thor. Surg., 64, 251 (1997); D. I. Walker et al., Brit. J. Surg., 59, 609 (1972); R. L. Pennell et al., J. Vasc. Surg., 2, 859 (1985)), and restenosis following balloon angioplasty (see, R. Ross cited above). The cells involved with inflammation include leukocytes (i.e., the immune system cells-neutrophils, eosinophils, lymphocytes, monocytes, basophils, macrophages, dendritic cells, and mast cells), the vascular endothelium, vascular smooth muscle cells, fibroblasts, and myocytes.

The release of inflammatory cytokines such as tumor necrosis factor-alpha (TNF-α) by leukocytes is a means by which the immune system combats pathogenic invasions, including infections. TNF-α stimulates the expression and activation of adherence factors on leukocytes and endothelial cells, primes neutrophils for an enhanced inflammatory response to secondary stimuli and enhances adherent neutrophil oxidative activity (see, Sharma et al., cited above). In addition, macrophages/dendritic cells act as accessory cells processing antigen for presentation to lymphocytes. The lymphocytes, in turn, become stimulated to act as pro-inflammatory cytotoxic cells.

Generally, cytokines stimulate neutrophils to enhance oxidative (e.g., superoxide and secondary products) and nonoxidative (e.g., myeloperoxidase and other enzymes) inflammatory activity. Inappropriate and over-release of cytokines can produce counterproductive exaggerated pathogenic effects through the release of tissue-damaging oxidative and nonoxidative products (K. G. Tracey et al., J. Exp. Med., 167, 1211 (1988); and D. N. Mannel et al., Rev. Infect. Dis., 9 (suppl. 5), S602-S606 (1987)). For example, TNF-α can induce neutrophils to adhere to the blood vessel wall and then to migrate through the vessel to the site of injury and release their oxidative and non-oxidative inflammatory products.

Although monocytes collect slowly at inflammatory foci, given favorable conditions, the monocytes develop into long-term resident accessory cells and macrophages. Upon stimulation with an inflammation trigger, monocytes/macrophages also produce and secrete an array of cytokines (including TNF-α), complement, lipids, reactive oxygen species, proteases and growth factors that remodel tissue and regulate surrounding tissue functions.

For example, inflammatory cytokines have been shown to be pathogenic in: arthritis (C. A. Dinarello, Semin. Immunol., 4, 133 (1992)); ischemia (A. Seekamp et al., Agents-Actions-Supp., 41, 137 (1993)); septic shock (D. N. Mannel et al., Rev. Infect. Dis., 9 (suppl. 5), S602-S606 (1987)); asthma (N. M. Cembrzynska et al., Am. Rev. Respir. Dis., 147, 291 (1993)); organ transplant rejection (D. K. Imagawa et al., Transplantation, 51, 57 (1991); multiple sclerosis (H. P. Hartung, Ann. Neurol., 33, 591 (1993)); AIDS (T. Matsuyama et al., AIDS, 5, 1405 (1991)); and in alkali-burned eyes (F. Miyamoto et al., Opthalmic Res., 30, 168 (1997)). In addition, superoxide formation in leukocytes has been implicated in promoting replication of the human immunodeficiency virus (HIV) (S. Legrand-Poels et al., AIDS Res. Hum. Retroviruses, 6, 1389 (1990)).

It is well known that adenosine and some analogs of adenosine that nonselectively activate adenosine receptor subtypes decrease neutrophil production of inflammatory oxidative products (B. N. Cronstein et al., Ann. N.Y. Acad. Sci., 451, 291 (1985); P. A. Roberts et al., Biochem. J., 227, 669 (1985); D. J. Schrier et al., J. Immunol., 137, 3284 (1986); B. N. Cronstein et al., Clinical Immunol. and Immunopath., 42, 76 (1987); M. A. Iannone et al., in Topics and Perspective in Adenosine Research, E. Gerlach et al., eds., Springer-Verlag, Berlin, p. 286 (1987); S. T. McGarrity et al., J. Leukocyte Biol., 44, 411-421 (1988); J. De La Harpe et al., J. Immunol., 143, 596 (1989); S. T. McGarrity et al., J. Immunol., 142, 1986 (1989); and C. P. Nielson et al., Br. J. Pharmacol., 97, 882 (1989)). For example, adenosine has been shown to inhibit superoxide release from neutrophils stimulated by chemoattractants such as the synthetic mimic of bacterial peptides, f-met-leu-phe (fMLP), and the complement component C₅ a (B. N. Cronstein et al., J. Immunol., 135, 1366 (1985)). Adenosine can decrease the greatly enhanced oxidative burst of PMN (neutrophil) first primed with TNF-α and then stimulated by a second stimulus such as f-met-leu-phe (G. W. Sullivan et al., Clin. Res., 41, 172A (1993)). Additionally, it has been reported that adenosine can decrease the rate of HIV replication in a T-cell line (S. Sipka et al., Acta. Biochim. Biopys. Hung., 23, 75 (1988)). However, there is no evidence that in vivo adenosine has anti-inflammatory activity (G. S. Firestein et al., Clin. Res., 41, 170A (1993); and B. N. Cronstein et al., Clin. Res., 41, 244A (1993)).

It has been suggested that there is more than one subtype of adenosine receptor on neutrophils that can have opposite effects on superoxide release (B. N. Cronstein et al., J. Clin. Invest., 85, 1150 (1990)). The existence of A_{2A} receptor on neutrophils was originally demonstrated by Van Calker et al. (D. Van Calker et al., Eur. J. Pharmacology, 206, 285 (1991)).

There has been progressive development of compounds that are more and more potent and/or selective as agonists of A_{2A} adenosine receptors based on radioligand binding assays and physiological responses. Initially, compounds with little or no selectivity for A_{2A} receptors were developed, such as adenosine itself or 5′-carboxamides of adenosine, such as 5′-N-ethylcarboxamidoadenosine (NECA) (B. N. Cronstein et al., J. Immunol., 135, 1366 (1985)). Later, it was shown that addition of 2-alkylamino substituents increased potency and selectivity, e.g., CV1808 and CGS21680 (M. F. Jarvis et al., J. Pharmacol. Exp. Ther., 251, 888 (1989)). 2-Alkoxy-substituted adenosine derivatives such as WRC-0090 are even more potent and selective as agonists at the coronary artery A_{2A} receptor (M. Ueeda et al., J. Med. Chem., 34, 1334 (1991)). The 2-alklylhydrazino adenosine derivatives, e.g., SHA 211 (also called WRC-0474) have also been evaluated as agonists at the coronary artery A_{2A} receptor (K. Niiya et al., J. Med. Chem., 35, 4557 (1992)).

A continuing need exists for selective A_{2A} adenosine receptor agonists and their combinations with other therapeutic agents such as anti-inflammatory compounds to provide specific and effective compositions and methods for treatment of inflammatory disorders which overcome the deficiencies of the prior art compositions and methods.

### SUMMARY OF THE INVENTION

The present invention comprises methods for the treatment of inflammatory activity in mammalian tissue. The inflammatory tissue activity can be due to pathological agents or can be due to physical, chemical, or thermal trauma, or the trauma of medical procedures, such as organ, tissue or cell transplantation, angioplasty (PCTA), inflammation following ischemia/reperfusion, or grafting. Thus, the present invention provides a method for inhibiting the inflammatory response in a mammal, such as a human subject, and protecting the tissue subject to the response, by administering an effective amount of a combination of at least one A_{2A} adenosine receptor agonist and at least one anti-inflammatory compound. By coadministering an anti-inflammatory compound with the agonist of the A_{2A} adenosine receptor, it is possible to dramatically lower the dosage of the A_{2A} adenosine receptor agonist and the anti-inflammatory compound due to a synergistic effect of the two agents. This reduces the possibility of side effects.

### DETAILED DESCRIPTION OF THE INVENTION

The following definitions are used, unless otherwise described. Halo is fluoro, chloro, bromo, or iodo. Alkyl, alkoxy, aralkyl, alkylaryl, etc. denote both straight and branched alkyl groups; but reference to an individual radical such as “propyl” embraces only the straight chain radical, a branched chain isomer such as “isopropyl” being specifically referred to. Aryl includes a phenyl radical or an ortho-fused bicyclic carbocyclic radical having about nine to ten ring atoms in which at least one ring is aromatic. Heteroaryl includes a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, or enzymatic techniques, by synthesis from optically-active starting materials, by chiral synthesis, or by chromatographic separation using a chiral stationary phase) and how to determine adenosine agonist activity using the tests described herein, or using other similar tests which are well known in the art.

Specific and preferred values listed below for radicals, substituents, and ranges, are for illustration only; they do not exclude other defined values or other values within defined ranges for the radicals and substituents.

(C₁-C₆)alkyl includes methyl, ethyl, propyl, isopropyl, butyl, iso-butyl, sec-butyl, pentyl, 3-pentyl, or hexyl.

Cycloalkyl includes bicycloalkyl (norbornyl, 2.2.2-bicyclooctyl, etc.) and tricycloalkyl (adamantyl, etc.), optionally comprising 1-2 N, O, or S. Cycloalkyl also encompasses (cycloalkyl)alkyl. Examples of (C₃-C₆)cycloalkyl include cyclopropyl, cyclobutyl, cyclopentyl, and cyclohexyl. (C₃-C₆)cycloalkyl(C₁-C₆)alkyl includes cyclopropylmethyl, cyclobutylmethyl, cyclopentylmethyl, cyclohexylmethyl; 2-cyclopropylethyl, 2-cyclobutylethyl, 2-cyclopentylethyl, and 2-cyclohexylethyl.

(C₁-C₆)alkoxy includes methoxy, ethoxy, propoxy, isopropoxy, butoxy, iso-butoxy, sec-butoxy, pentoxy, 3-pentoxy, or hexyloxy.

(C₂-C₆)alkenyl includes vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl.

(C₂-C₆)alkynyl includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl.

(C₁-C₆)alkanoyl includes acetyl, propanoyl, and butanoyl.

Halo(C₁-C₆)alkyl includes iodomethyl, bromomethyl, chloromethyl, fluoromethyl, trifluoromethyl, 2-chloroethyl, 2-fluoroethyl, 2,2,2-trifluoroethyl, and pentafluoroethyl.

Hydroxy(C₁-C₆)alkyl includes hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, and 6-hydroxyhexyl.

(C₁-C₆)alkoxycarbonyl (CO₂R²) includes methoxycarbonyl, ethoxycarbonyl, propoxycarbonyl, isopropoxycarbonyl, butoxycarbonyl, pentoxycarbonyl, or hexyloxycarbonyl.

(C₁-C₆)alkylthio includes methylthio, ethylthio, propylthio, isopropylthio, butylthio, isobutylthio, pentylthio, and hexylthio.

(C₂-C₆)alkanoyloxy includes acetoxy, propanoyloxy, butanoyloxy, isobutanoyloxy, pentanoyloxy, and hexanoyloxy.

Aryl includes phenyl, indenyl, and naphthyl.

Heteroaryl includes furyl, imidazolyl, triazolyl, triazinyl, oxazoyl, isoxazoyl, thiazolyl, isothiazoyl, pyraxolyl, pyrrolyl, pyrazinyl, tetrazolyl, puridyl (or its N-oxide), thientyl, pyrimidinyl (or its N-oxide), indolyl, isoquinolyl (or its N-oxide), and quinolyl (or its N-oxide).

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting its development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

The present invention provides a method comprising treating an inflammatory response in a mammal in need of such treatment by administering an effective amount of an anti-inflammatory compound in combination with an effective amount of a compound having formula (I):

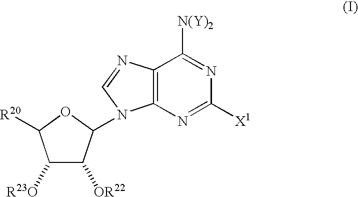

wherein:

X¹ is selected from —OR¹, —NR²R³, —C═C—Z, and —NH—N═R⁴;

R¹ is selected from:

(a) C₁₋₄ alkyl;

(b) C₁₋₄ alkyl substituted with one or more C₁₋₄ alkoxy, halogen, hydroxy, amino, mono(C₁₋₄ alkyl)amino, di(C₁₋₄ alkyl)amino, and C₆₋₁₀ aryl, wherein aryl may be substituted with one or more halogen, C₁₋₄ alkyl, hydroxy, amino, mono(C₁₋₄ alkyl)amino, di(C₁₋₄ alkyl)amino, and R¹⁵OOC—C₁₋₄ alkyl-;

(c) C₆₋₁₀ aryl; and

(d) C₆₋₁₀ aryl substituted with one or more halogen, hydroxy, amino, mono(C₁₋₄ alkyl)amino, di(C₁₋₄ alkyl)amino, and C₁₋₄ alkyl;

one of R² and R³ has the same meaning as R¹ and the other is hydrogen;

R⁴ is a group having the formula:

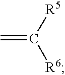

R⁵ and R⁶ are independently selected from H, C₃₋₇-cycloalkyl, or any of the meanings of R¹, provided that R⁵ and R⁶ are not both hydrogen;

Z is selected from (a)-(e):

(a) phenyl or naphthyl optionally substituted with one to three groups selected from halogen, C₁₋₆ alkyl, C₁₋₆ haloalkyl, C₁₋₆ alkoxy, C₁₋₆ haloalkoxy, C₂₋₆ alkoxycarbonyl, C₂₋₆ alkoxyalkyl, C₁₋₆ alkylthio, thio, CHO, cyanomethyl, nitro, cyano, hydroxy, carboxy, C₂₋₆ acyl, amino, C₁₋₃ monoalkylamino, C₂₋₆ dialkylamino, methylenedioxy; and aminocarbonyl;

(b) a group of formula —(CH₂)ₘ-Het, wherein m is 0 or an integer from 1 to 3 and Het is 5-6 membered heterocyclic aromatic or non-aromatic ring, optionally benzo condensed, containing 1-3 heteroatoms selected from nonperoxide oxygen, nitrogen, and sulfur, linked through a carbon atom or through a nitrogen atom;

(c) C₂-C₄ alkenyl or C₃-C₇ cycloalkyl optionally containing unsaturation;

(d)

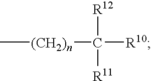

and,

(e) C₁-C₁₆ alkyl, optionally comprising 1-2 double bonds, O, S or NY;

R¹⁰ is selected from H, methyl, and phenyl;

R¹² is selected from H, C₁-C₆ alkyl, C₅-C₆ cycloalkyl, C₃-C₇ cycloalkenyl, and phenyl-C₁₋₂ alkyl-;

alternatively, R¹⁰ and R¹², taken together, form a 5- or 6-membered carbocyclic ring;

alternatively, R³ is hydrogen and R² and R⁴, taken together, form an oxo group or a corresponding acetalic derivative;

R¹¹ is selected from OH, NH₂ dialkylamino, halogen, and cyano;

n is selected from 0, 1, 2, 3, and;

Y is individually selected from H, C₁₋₆ alkyl, C₃₋₇ cycloalkyl, phenyl, and phenyl-C₁₋₃ alkyl-;

R²⁰ is selected from —C(═O)NR¹⁶R¹⁷, COOR¹⁵, and CH₂OR¹⁵;

each of R¹⁶ and R¹⁷ is independently selected from:

(a) H;

(b) C₃₋₇ cycloalkyl;

(c) C₁₋₄ alkyl;

(d) C₁₋₄ alkyl substituted with one or more C₁₋₄ alkoxy, halogen, hydroxy, —COOR²¹, amino, mono(C₁₋₄ alkyl)amino, di(C₁₋₄ alkyl)amino or C₆₋₁₀ aryl, wherein aryl is optionally substituted with one or more groups selected from halogen, C₁₋₄ alkyl, hydroxy, amino, mono(C₁₋₄ alkyl)amino, di(C₁₋₄ alkyl)amino, and R¹⁵OOC—C₁₋₄ alkyl-;

(e) C₆₋₁₀ aryl; and,

(f) C₆₋₁₀ aryl substituted with one or more halogen, hydroxy, amino, mono(C₁₋₄ alkyl)amino, di(C₁₋₄ alkyl)amino, and C₁₋₄ alkyl;

R²² and R²³ independently represent H, C₁₋₆ alkanoyl, C₁₋₆ alkoxyC₁₋₆ alkanoyl-, aroyl, carbamoyl, and mono or diC₁₋₆ alkylcarbamoyl;

R¹⁵ is selected from H, C₁₋₄ alkyl, C₆₋₁₀ aryl, and C₆₋₁₀ aryl-C₁₋₄ alkyl-; and

R²¹ is independently selected from hydrogen C₁₋₄ alkyl, C₆₋₁₀ aryl, and C₆₋₁₀ aryl-C₁₋₄ alkyl-;

or a pharmaceutically acceptable salt thereof.

The present invention also provides methods, wherein both R²² and R²³ represent hydrogen.

The present invention also provides methods, wherein R³ is C₁₋₄ alkyl substituted with C₆₋₁₀ aryl, wherein aryl is substituted with R¹⁵OOC—C₁₋₄ alkyl-.

The present invention also provides methods, wherein one of R¹⁶ and R¹⁷ is C₁₋₄-alkyl substituted with one or more groups selected from C₁₋₄ alkoxy, halogen, hydroxy, amino, mono(C₁₋₄ alkyl)amino, di(C₁₋₄ alkyl)amino, and C₆₋₁₀ aryl, wherein aryl is optionally substituted with one or more groups selected from halogen, hydroxy, amino, C₁₋₄ alkyl, mono(C₁₋₄ alkyl)amino or di(C₁₋₄ alkyl)amino, and R¹⁵OOC—C₁₋₄ alkyl-.

The present invention also provides methods, wherein one of R¹⁶ and R¹⁷ is C₆₋₁₀ aryl substituted with one or more groups selected from halogen, hydroxy, amino, mono(C₁₋₄ alkyl)amino, di(C₁₋₄ alkyl)amino, and C₁₋₄ alkyl.

The present invention also provides methods, wherein R¹⁶ is H and R¹⁷ is selected from C₁₋₄ alkyl, cyclopropyl, and hydroxy-C₂₋₄ alkyl.

The present invention also provides methods, wherein R²⁰ is ethylaminocarbonyl.

The present invention also provides methods, wherein X¹ is —NR²R³.

The present invention also provides methods, wherein R² is H and R³ is selected from C₁₋₄ alkyl substituted with C₆₋₁₀ aryl substituted with R¹⁵OOC—C₁₋₄ alkyl-.

The present invention also provides methods, wherein R¹⁵ is selected from H, methyl, ethyl, n-propyl, isopropyl, and tert-butyl.

The present invention also provides methods, wherein R¹⁵ is selected from H, methyl, and ethyl.

The present invention also provides methods, wherein R¹⁵ is methyl.

The present invention also provides methods, wherein R¹⁵ is H.

The present invention also provides methods, wherein the adenosine A_{2A} receptor agonist is selected from a group consisting of:

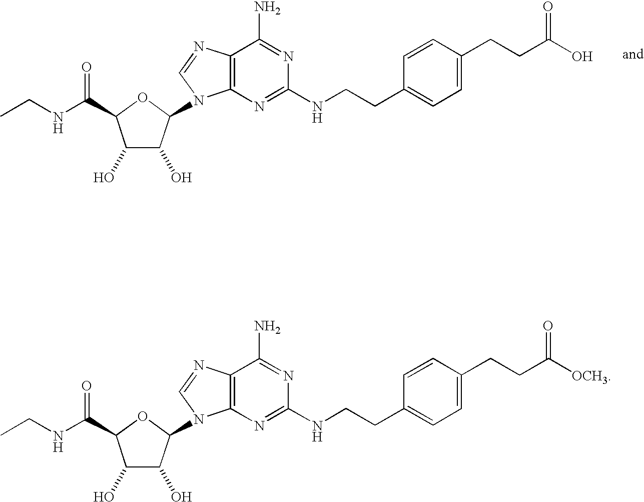

The present invention also provides methods, wherein X¹ is —C≡C—Z.

The present invention also provides methods, wherein the adenosine A_{2A} receptor agonist is selected from a group consisting of:

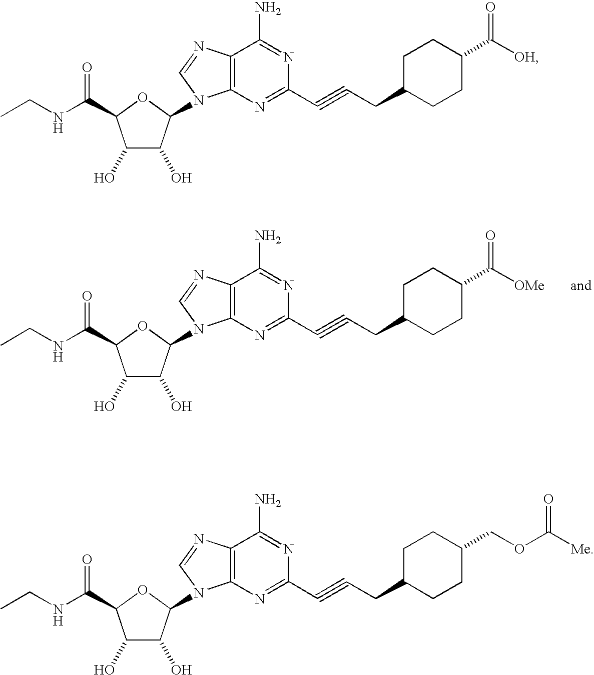

The present invention also provides methods, wherein R²⁰ is —CH₂OR¹⁵.

The present invention also provides methods, wherein R¹⁵ is hydrogen.

The present invention also provides methods, wherein X¹ is —NH—N═R⁴.

The present invention also provides methods, wherein the adenosine A_{2A} receptor agonist is selected from a group consisting of:

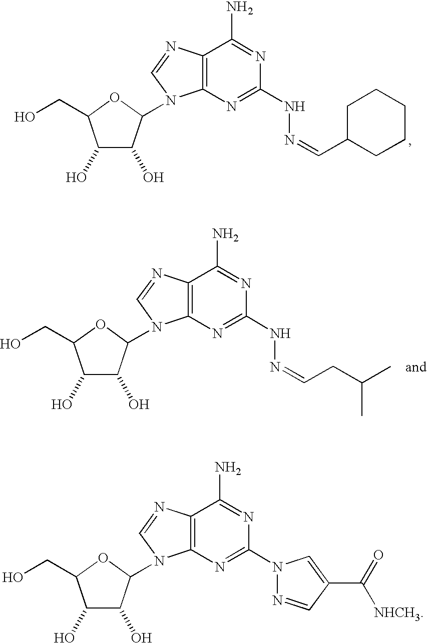

The present invention also provides methods, wherein X¹ is —OR¹.

The present invention also provides methods, wherein the adenosine A_{2A} receptor agonist is selected from a group consisting of:

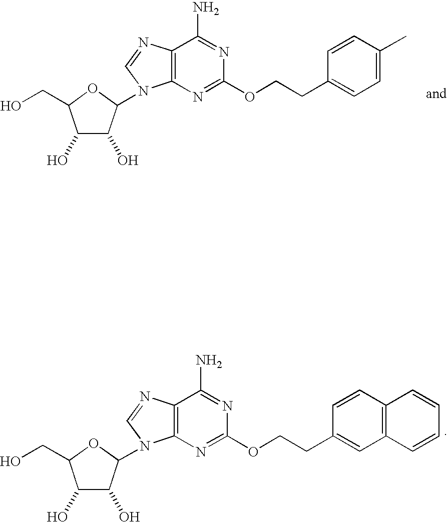

The compounds described herein may be synthesized as described in: Olsson et al. (U.S. Pat. Nos. 5,140,015 and 5,278,150); Cristalli (U.S. Pat. No. 5,593,975); Miyasaka et al. (U.S. Pat. No. 4,956,345); Hutchinson, A. J. et al., J. Pharmacol. Exp. Ther., 251, 47 (1989); Olsson, R. A. et al., J. Med. Chem., 29, 1683 (1986); Bridges, A. J. et al., J. Med. Chem., 31, 1282 (1988); Hutchinson, A. J. et al., J. Med. Chem., 33, 1919 (1990); Ukeeda, M. et al., J. Med. Chem., 34, 1334 (1991); Francis, J. E. et al., J. Med. Chem., 34, 2570 (1991); Yoneyama, F. et al., Eur. J. Pharmacol., 213, 199-204 (1992); Peet, N. P. et al., J. Med. Chem., 35, 3263 (1992); and Cristalli, G. et al., J. Med. Chem., 35, 2363 (1992); all of which are incorporated herein by reference.

Compositions and Methods

Compositions and methods are provided that are effective for treating a wide variety of inflammatory disorders. The compositions comprise a combination of an effective amount of at least one adenosine A_{2A} receptor agonist (referred to hereafter as an A_{2A} agonist) and an effective amount of at least one anti-inflammatory compound that is not a phosphodiesterase-4 inhibitor (hereafter PDE4 inhibitor). The methods comprise treating a subject, advantageously a human subject, suffering from an inflammatory disorder, with a composition comprising a combination of an effective amount of at least one adenosine A_{2A} agonist and an effective amount of at least one anti-inflammatory compound that is not a PDE4 inhibitor. The present inventors have discovered that the combination of an adenosine A_{2A} agonist and an anti-inflammatory compound provides unexpectedly superior properties as compared to either compound alone.

Prior work has suggested combining adenosine A_{2A} agonists with PDE4 inhibitors. The rationale for making this combination lies in the complementary modes of action of A_{2A} agonists and PDE4 inhibitors: A_{2A} agonists act by stimulating the production of cyclic AMP in inflammatory cells while PDE4 inhibitors inhibit cyclic AMP degradation in inflammatory cells. Both compounds therefore act directly to increase cyclic AMP production and the expectation is that the combination will have enhanced anti-inflammatory activity. The present application is directed to combinations of A_{2A} agonists and anti-inflammatory compounds that are not PDE4 inhibitors and that are not known to have the same direct effects on cyclic AMP production as PDE4 inhibitors.

Specific examples of adenosine A_{2A} agonists that can be used in the present invention are described in detail below. The skilled artisan will recognize, however, that the invention is not limited to combinations containing the specific A_{2A} agonists that are described below, but extends to combinations of any A_{2A} agonist that is presently known or that is discovered in the future. Similarly, although suitable anti-inflammatory compounds are described in detail below, the skilled artisan will recognize that these compounds are merely exemplary and that the invention extends to all anti-inflammatory compounds, other than compounds that are known to be PDE4 inhibitors, that presently are known or that are discovered in the future.

The adenosine A_{2A} receptor agonist as mentioned above can be combined with at least one anti-inflammatory compound. The present invention also includes the administration of at least one anti-inflammatory compound in combination with at least one A_{2A} adenosine receptor agonist as described above.

The anti-inflammatory compound can be selected from the group consisting of the following:

(a) Leukotriene biosynthesis inhibitors, 5-lipoxygenase (5-LO) inhibitors, and 5-lipoxygenase activating protein (FLAP) antagonists, including zileuton; ABT-761; fenleuton; tepoxalin; Abbott-79175; Abbott-85761; N-(5-substituted)-thiophene-2-alkylsulfonamides; 2,6-di-tert-butylphenol hydrazones; Zeneca ZD-2138; SB-210661; pyridinyl-substituted 2-cyanonaphthalene compound L-739,010; 2-cyanoquinoline compound L-746,530; indole and quinoline compounds MK-591, MK-886, and BAY x 1005;

(b) Receptor antagonists for leukotrienes LTB₄, LTC₄, LTD₄, and LTE₄, including phenothiazin-3-one compound L-651,392; amidino compound CGS-25019c; benzoxazolamine compound ontazolast; benzenecarboximidamide compound BIIL 284/260; compounds zafirlukast, ablukast, montelukast, pranlukast, verlukast (MK-679), RG-12525, Ro-245913, iralukast (CGP 45715A), and BAY x 7195;

(c) 5-Lipoxygenase (5-LO) inhibitors; and 5-lipoxygenase activating protein (FLAP) antagonists;

(d) Dual inhibitors of 5-lipoxygenase (5-LO) and antagonists of platelet activating factor (PAF);

(e) Leukotriene antagonists (LTRAs) of LTB₄, LTC₄, LTD₄, and LTE₄;
(f) Antihistaminic H₁ receptor antagonists, including cetirizine, loratadine, desloratadine, fexofenadine, astemizole, azelastine, and chlorpheniramine;
(g) Gastroprotective H₂ receptor antagonists;
(h) α₁- and α₂-adrenoceptor agonist vasoconstrictor sympathomimetic agents administered orally or topically for decongestant use, including propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride;
(i) one or more α₁- and α₂-adrenoceptor agonists as recited in (h) above in combination with one or more inhibitors of 5-lipoxygenase (5-LO) as recited in (a) above;
(j) Theophylline and aminophylline;
(k) Sodium cromoglycate;
(l) Muscarinic receptor (M1, M2, and M3) antagonists;
(m) COX-1 inhibitors (NTHEs); and nitric oxide NTHEs;
(n) COX-2 selective inhibitor for example rofecoxib and celecoxib;
(o) COX-3 inhibitor for example acetaminophen;
(p) insulin-like growth factor type I (IGF-1) mimetics;
(q) Ciclesonide;
(r) Corticosteroids, including prednisone, methylprednisone, triamcinolone, beclomethasone, fluticasone, budesonide, hydrocortisone, dexamethasone, mometasone furoate, azmacort, betamethasone, beclovent, prelone, prednisolone, flunisolide, triamcinolone acetonide, beclomethasone dipropionate, fluticasone propionate, mometasone furoate, solumedrol and salmeterol;
(s) Tryptase inhibitors;
(t) Platelet activating factor (PAF) antagonists;
(u) Monoclonal antibodies active against endogenous inflammatory entities;
(v) IPL 576;
(w) Anti-tumor necrosis factor (TNF-α) agents, including etanercept, infliximab, and D2E7;
(x) DMARDs for example leflunomide;
(y) Elastase inhibitors, including UT-77 and ZD-0892;
(z) TCR peptides;
(aa) Interleukin converting enzyme (ICE) inhibitors;
(bb) IMPDH inhibitors;
(cc) Adhesion molecule inhibitors including VLA-4 antagonists;
(dd) Cathepsins;
(ee) Mitogen activated protein kinase (MAPK) inhibitors;
(ff) Mitogen activated protein kinase kinase (MAPKK) inhibitors;
(gg) Glucose-6 phosphate dehydrogenase inhibitors;
(hh) Kinin-B₁- and B₂-receptor antagonists;
(ii) Gold in the form of an aurothio group in combination with hydrophilic groups;
(jj) Immunosuppressive agents, including cyclosporine, azathioprine, tacrolimus, and methotrexate;
(kk) Anti-gout agents, including colchicine;
(ll) Xanthine oxidase inhibitors, including allopurinol;
(mm) Uricosuric agents, including probenecid, sulfinpyrazone, and benzbromarone;
(nn) Antineoplastic agents that are antimitotic drugs for example vinblastine, vincristine, cyclophosphamide, and hydroxyurea;
(oo) Growth hormone secretagogues;
(pp) Inhibitors of matrix metalloproteinases (MMPs), including the stromelysins, the collagenases, the gelatinases, aggrecanase, collagenase-1 (MMP-1), collagenase-2 (MMP-8), collagenase-3 (MMP-13), stromelysin-1 (MMP-3), stromelysin-2 (MMP-10), and stromelysin-3 (MMP-11);
(qq) Transforming growth factor (TGFβ);
(rr) Platelet-derived growth factor (PDGF);
(ss) Fibroblast growth factor, including basic fibroblast growth factor (bFGF);
(tt) Granulocyte macrophage colony stimulating factor (GM-CSF);
(uu) Capsaicin; and
(vv) Tachykinin NK₁ and NK₃ receptor antagonists, including NKP-608C; SB-233412 (talnetant); and D-4418.

The anti-inflammatory compound and the adenosine receptor agonist may be administered either simultaneously or one after another in any order so as to be effective in inhibiting an inflammatory disorder.

Among the inflammatory disorders that can be treated (including treated prophylactically) are inflammation due to:

Among the inflammatory disorders that can be treated (including treated prophylactically) are inflammation due to the following.

(a) autoimmune stimulation (autoimmune diseases), including systemic lupus erythematosus, lupus nephritis, multiple sclerosis, Addison's disease, Myasthenia gravis, vasculitis (e.g. Wegener's granulomatosis), autoimmune hepatitis, infertility from endometriosis, type I diabetes mellitus including the destruction of pancreatic islets leading to diabetes and the inflammatory consequences of diabetes, including leg ulcers, Crohn's disease, ulcerative colitis, inflammatory bowel disease, osteoporosis and rheumatoid arthritis;

(b) allergic diseases including asthma, hay fever, rhinitis, vernal conjunctivitis and other eosinophil-mediated conditions;

(c) skin diseases including psoriasis, contact dermatitis, eczema, infectious skin ulcers, open wounds, cellulitis;

(d) infectious diseases including sepsis, septic shock, encephalitis, infectious arthritis, endotoxic shock, gram negative shock, Jarisch-Herxheimer reaction, shingles, toxic shock, cerebral malaria, bacterial meningitis, acute respiratory distress syndrome (ARDS), lyme disease, HIV infection, (TNF-α-enhanced HIV replication, TNF-α inhibition of reverse transcriptase inhibitor activity);

(e) wasting diseases: cachexia secondary to cancer and HIV;

(f) organ, tissue or cell transplantation (e.g., bone marrow, cornea, kidney, lung, liver, heart, skin, pancreatic islets) including transplant rejection, preeclampsia resulting from pregnancy, and acute or chronic flares of graft versus host disease;

(g) adverse effects from drug therapy, including adverse effects from amphotericin B treatment, adverse effects from immunosuppressive therapy, e.g., interleukin-2 treatment, adverse effects from OKT3 treatment, adverse effects from GM-CSF treatment, adverse effects of cyclosporine treatment, and adverse effects of aminoglycoside treatment, stomatitis and mucositis due to immunosuppression;

(h) cardiovascular conditions including circulatory diseases induced or exasperated by an inflammatory response, such as ischemia, atherosclerosis, peripheral vascular disease, restenosis following angioplasty, inflammatory aortic aneurysm, vasculitis, stroke, spinal cord injury, congestive heart failure, hemorrhagic shock, ischemia/reperfusion injury, vasospasm following subarachnoid hemorrhage, vasospasm following cerebrovascular accident, pleuritis, pericarditis, and the cardiovascular complications of diabetes;

(i) dialysis, including pericarditis, due to peritoneal dialysis;

(j) gout;

(k) chemical or thermal trauma due to burns, acid, and alkali; and,

(l) chemical poisoning (MPTP/concavalin/chemical agent/pesticide poisoning).

Also of particular interest and efficacy is the use of the present invention to treat inflammatory responses due to organ, tissue or cell transplantation, i.e., the transplantation of allogeneic or xenogeneic tissue into a mammalian recipient, inflammation due to autoimmune diseases and inflammatory responses due to circulatory pathologies such as ischemia, and the treatment thereof, including angioplasty, stent placement, shunt placement or grafting.

Also of particular interest and efficacy is the use of the present invention to treat inflammatory responses in situations where a short acting or rapidly metabolized compound is useful. Examples of such use include topical treatment of skin lesions or burns to promote local wound healing without causing systemic side effects.

The exact dosage of the compound of formula (I) to be administered will, of course, depend on the size and condition of the patient being treated, the exact condition being treated, and the identity of the particular compound of formula (I) being administered. However, a suitable dosage of the compound of formula (I) is 0.5 to 100 μg/kg of body weight, preferably 1 to 10 μg/kg of body weight. Typically, the compound of formula (I) will be administered from 1 to 8, preferably 1 to 4, times per day.

The preferred mode of administration of the compound of formula (I) may also depend on the exact condition being treated. However, most typically, the mode of administration will be oral, topical, intravenous, parenteral, subcutaneous, as an aerosol or by intramuscular injection.

It is to be understood that the compound of formula (I) may be administered in the form of a pharmaceutically acceptable salt. Examples of such salts include acid addition salts. Preferred pharmaceutically acceptable addition salts include salts of mineral acids, for example, hydrochloric acid, sulfuric acid, nitric acid, and the like; salts of monobasic carboxylic acids, such as, for example, acetic acid, propionic acid, and the like; salts of dibasic carboxylic acids, such as maleic acid, famaric acid, oxalic acid, and the like; and the salts of tribasic carboxylic acids, such as carboxysuccinic acid, citric acid, and the like. In the compounds of formula (I) in which R is —CO₂H, the salt may be derived by replacing the acidic proton of the —CO₂H group with a cation such as Na⁺, K⁺, NH₄⁺ mono-, di-, tri- or tetra(C₁₋₄ alkyl)ammonium or mono-, di-, tri- or tetra(C₂₋₄ alkanol) ammonium.

Preferred pharmaceutically acceptable addition salts include salts of mineral acids, for example, hydrochloric acid, sulfuric acid, nitric acid, and the like; salts of monobasic carboxylic acids, such as, for example, acetic acid, propionic acid, and the like; salts of dibasic carboxylic acids, such as maleic acid, fumaric acid, oxalic acid, and the like; and salts of tribasic carboxylic acids, such as, carboxysuccinic acid, citric acid, and the like.

It is also to be understood that many of the compounds of formula (I) may exist as various isomers, enantiomers, and diastereomers and that the present invention encompasses the administration of a single isomer, enantiomer or diastereomer in addition to the administration of mixtures of isomers, enantiomers or diastereomers.

The compounds of formula (I) can be administered orally, for example, with an inert diluent with an edible carrier. They can be enclosed in gelatin capsules or compressed into tablets. For the purposes of oral therapeutic administration, the compounds can be incorporated with excipients and used in the form of tablets, troches, capsules, elixers, suspensions, syrups, waters, chewing gums, and the like. These preparations should contain at least 0.5% by weight of the compound of formula (I), but the amount can be varied depending upon the particular form and can conveniently be between 4.0% to about 70% by weight of the unit dosage. The amount of the compound of formula (I) in such compositions is such that a suitable dosage will be obtained. Preferred compositions and preparations according to the present invention are prepared so that an oral dosage unit form contains between about 30 μg and about 5 mg, preferably between 50 to 500 μg, of active compound.

Other adenosine A_{2A} receptor agonists known to a person skilled in the art can be employed in the present invention. For example, suitable A_{2A} agonists include, but are not limited to, the agonists disclosed in U.S. Pat. No. 6,232,297, WO 99/67264, WO 03/029264, WO 00/23457, and WO 00/78779, which compounds are herein incorporated by reference.

The anti-inflammatory compound may be administered in the form of a pharmaceutical composition similar to those described above in the context of the compound of formula (I).

While dosage values will vary with the specific disease condition to be alleviated, good results are achieved when the anti-inflammatory compound is administered to a subject requiring such treatment as an effective oral, parenteral or intravenous dose as described below.

For oral administration, the amount of active agent per oral dosage unit usually is 0.1-20 mg, preferably 0.5-10 mg. The daily dosage is usually 0.1-50 mg, preferably 1-30 mg p.o. For parenteral application, the amount of active agent per dosage unit is usually 0.005-10 mg, preferably 0.01-5 mg. The daily dosage is usually 0.01-20 mg, preferably 0.02-5 mg i.v. or i.m.

With topical administration, dosage levels and their related procedures would be consistent with those known in the art, such as those dosage levels and procedures described in U.S. Pat. No. 5,565,462 to Eitan et al., which is incorporated herein by reference.

It is to be understood, however, that for any particular subject, specific dosage regimens should be adjusted to the individual need and the professional judgment of the person administering or supervising the administration of the anti-inflammatory compound. In some cases, the compound of formula (I) will be administered for an extended period of time following the inflammatory insult, even chronically. It is to be further understood that the dosages set forth herein are exemplary only and that they do not, to any extent, limit the scope or practice of the present invention.

In a particularly preferred embodiment, the compound of formula (I) and the anti-inflammatory compound are combined together in a single dosage unit. The compound of formula (I) and the anti-inflammatory compound may be administered in the same type of pharmaceutical composition as those described above in the context of the compound of formula (I).

By coadministering an anti-inflammatory compound with the agonist of the A_{2A} adenosine receptor, it is possible to dramatically lower the dosage of the A_{2A} adenosine receptor agonist and the anti-inflammatory compound due to a synergistic effect of the two agents. Thus, in the embodiment involving coadministration of the A_{2A} adenosine receptor agonist with the anti-inflammatory compound, the dosage of the A_{2A} adenosine receptor agonist may be reduced by a factor of 5 to 10 from the dosage used when no anti-inflammatory compound is administered. This reduces the possibility of side effects.

The compounds of the invention can be administered by inhalation from an inhaler, insufflator, atomizer or pressurized pack or other means of delivering an aerosol spray. Pressurized packs may comprise a suitable propellant such as carbon dioxide or other suitable gas. In case of a pressurized aerosol, the dosage unit may be determined by providing a value to deliver a metered amount. The inhalers, insufflators, atomizers are fully described in pharmaceutical reference books such as Remington's Pharmaceutical Sciences 16^{th} ed. (1980) or 18th ed. (1990) Mack Publishing Co.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Tablets, pills, capsules, troches, and the like can contain the following ingredients: a binder, such as microcrystalline cellulose, gum tragacanth or gelatin; an excipient, such as starch or lactose; a disintegrating agent, such as alginic acid, Primogel, corn starch, and the like; a lubricant, such as magnesium stearate or Sterotes; a glidant, such as colloidal silicon dioxide; a sweetening agent, such as sucrose, saccharin or aspartame; or flavoring agent, such as peppermint, methyl salicylate or orange flavoring. When the dosage unit form is a capsule it can contain, in addition to the compound of formula (I), a liquid carrier, such as a fatty oil.

Other dosage unit forms can contain other materials that modify the physical form of the dosage unit, for example, as coatings. Thus, tablets or pills can be coated with sugar, shellac or other enteric coating agents. A syrup may contain, in addition to the active compounds, sucrose as a sweetening agent and preservatives, dyes, colorings, and flavors. Materials used in preparing these compositions should be pharmaceutically pure and non-toxic in the amounts used.

For purposes of parenteral therapeutic administration, the compounds of formula (I) can be incorporated into a solution or suspension. These preparations should contain at least 0.1% of the compound, but may be varied between 0.5% and about 50% of the weight thereof. The amount of active compound in such compositions is such that a suitable dosage will be obtained. Preferred compositions and preparations according to the present invention are prepared so that a parenteral dosage unit contains between 30 μg to 5 mg, preferably between 50 to 500 μg, of the compound of formula (I).

Solutions or suspensions of the compounds of formula (I) can also include the following components: a sterile diluent, such as water for injection, saline solution, fixed oils, polyethylene glycols, glycerine, propylene glycol or other synthetic solvents: antibacterial agents, such as benzyl alcohol or methyl parabens; antioxidants, such as ascorbic acid or sodium bisulfite; chelating agents, such as ethylenediaminetetraacetic acid; buffers, such as acetates, citrates or phosphates; and agents for the adjustment of tonicity, such as sodium chloride or dextrose. The parenteral preparation can be enclosed in ampoules, disposable syringes or multiple dose vials made of glass or plastic.

Effective amounts of the anti-inflammatory compound can be administered to a subject by any one of various methods, for example, orally as in a capsule or tablets, topically or parenterally in the form of sterile solutions. The anti-inflammatory compound, while effective themselves, can be formulated and administered in the form of their pharmaceutically acceptable addition salts for purposes of stability, convenience of crystallization, increased solubility, and the like.

### EXAMPLES

### Materials and Methods

Materials. f-Met-Leu-Phe (fMLP), luminol, and trypan blue were from Sigma Chemical. Ficoll-Hypaque was purchased from Flow Laboratories (McLean, A) and Los Alamos Diagnostics (Los Alamos, N.M.). Hanks balanced salt solution (HBSS), and limulus amebocyte lysate assay kit were from Whittaker Bioproducts (Walkersville, Md.). Human serum albumin (HSA) was from Cutter Biological (Elkhart, Ind.). Recombinant human tumor necrosis factor-alpha was supplied by Dianippon Pharmaceutical Co. Ltd. (Osaka, Japan). ZM241385 was a gift of Dr. Simon Poucher, Zeneca Pharmaceuticals (Chesire, England).

Leukocyte Preparation: Purified PMN (about 98% PMN and >95% viable by trypan blue exclusion) containing <1 platelet per 5 PMN and <50 pg/ml endotoxin (limulus amebocyte lysate assay) were obtained from normal heparinized (10 Units/ml) venous blood by a one-step Ficoll-Hypaque separation procedure (Ferrante, A. et al., J. Immunol. Meth., 36, 109 (1980)). Residual RBC were lysed by hypotonic lysis with iced 3 ml 0.22% sodium chloride solution for 45 seconds followed by 0.88 ml of 3% sodium chloride solution.

Chemiluminescence: Luminol -enhanced chemiluminescence, a measure of neutrophil oxidative activity, is dependent upon both superoxide production and mobilization of the granule enzyme myeloperoxidase. The light is emitted from unstable high-energy oxygen species generated by activated neutrophils. Purified PMN (5×10⁵/ml) were incubated in HBSS containing 0.1% human serum albumin (1 ml) with or without adenosine, adenosine analogs, and TNF-α (1 U/mL) for 30 minutes at 37° C. in a shaking water bath. Then luminol (1×10⁻⁴ M) enhanced f-met-leu-phe (1 μM) stimulated chemiluminescence was read with a Chronolog Photometer (Chrono-log Corp., Havertown, Pa.) at 37° C. for 8 min. Chemiluminescence is reported as relative peak light emitted (=height of the curve) compared to samples with TNF and without adenosine or adenosine analogs. WRC-0474[SHA 211] was 10 times more potent than either adenosine (ADO) or CGS21680 in decreased TNF-α-primed f-met-leu-phe-stimulated PMN chemiluminescence.

Synthesis of 3-(4-{2-[6-Amino-9-(5-ethylcarbamoyl-3,4-dihydroxy-tetrahydro-furan-2-yl)9H-purin-2-ylamino]-ethyl}-phenyl)-propionic acid methyl ester (JR2171): The methyl ester, JR2171, was synthesized by dissolving approximately 10.0 mg of 3-(4-{2-[6-amino-9-(5-ethylcarbamoyl-3,4-dihydroxy-tetrahydro-furan-2-yl)-9H-purin-2-ylamino]-ethyl}-phenyl)-propionic acid (CGS21680) in 5 mL 10% MeOH/CH₂Cl₂ and adding TMS Diazomethane (2.0 M solution in hexanes) dropwise until a yellow color persisted. After stirring for 30 minutes the solvent was removed under reduced pressure. The crude compound was then purified by HPLC using a C₁₈ column and a MeOH/H₂O gradient, yielding 6.7 mg of compound (66% yield). Characterization by HRMS, APCI MS, ¹H NMR, and ¹³C NMR were consistent with the putative structure.

Characterization Data:

High Resolution MS (HRMS) analysis performed by the University of Nebraska Center for Mass Spectrometry: actual=514.2408: calculated=514.2414

APCI MS data: Molecular ion=514.3

¹H NMR (CD₃OD) δ 7.93 (s, 1H), 7.09 (m, 4H), 5.88 (d, 1H), 4.95 (m, 1H), 4.42 (m, 1H), 4.33 (d, 1H), 3.58 (s, 3H), 3.43 (m, 2H), 3.07 (m, 2H), 2.80 (m, 4H), 2.54 (t, 2H), 0.96 (t, 3H).

¹³C NMR (CD₃OD) δ 173.2, 170.2, 159.3, 155.6, 155.5, 137.9, 137.3, 137.1, 128.2, 127.6, 112.8, 88.2, 83.7, 72.8, 71.3, 50.2, 42.4, 34.8, 34.7, 33.3, 29.7, 12.8.

Synergy of A_{2A} Adenosine Receptor Agonist and corticosteroids: The synergy between WRC-0474[SHA 211] and 4-(3-cyclopentyloxy-4-methoxyphenyl)-2-pyrrolidone (a corticosteroid) was examined by measuring the effect of combined WRC-0474[SHA 211] and a corticosteroid on TNF-primed f-met-leu-phe-stimulated suspended neutrophil superoxide release and on the oxidative burst of neutrophils adhering to matrix proteins (in this model, the PMN oxidative burst is enhanced by small concentrations of TNF-α [e.g., 1 U/ml] when added prior to the addition of a second stimulus such as the peptide f-met-leu-phe).

Suspended PMN Superoxide Release. Human PMN (1×10⁶/ml) from Ficoll-Hypaque separation were primed for 30 minutes (37° C.) with or without rhTNF (10 U/ml), with adenosine deaminase (1 U/ml), and with or without 4-(3-cyclopentyloxy-4-methoxyphenyl)-2-pyrrolidone and SHA 211. Cytochrome c (120 μM), catalase (0.062 mg/ml) and fMLP (100 nM) were added and the samples incubated for 10 minutes more at 37° C. SOD (200 U/ml) was added to matched samples. The samples were iced and centrifuged (2000 g×10 minutes). The optical density of the supernatants was read at 550 nm against the matched SOD samples, and the nmoles of SOD-inhibitable superoxide released in 10 minutes were calculated.

TNF-α-stimulated superoxide release of PMN adherent to a matrix protein (fibrinogen) coated surface: Human PMN (1×10⁶/ml) from Ficoll-Hypaque separation were incubated for 90 minutes in 1 ml of Hanks balanced salt solution containing 0.1% human serum albumin, cytochrome c (120 μM), and catalase (0.062 mg/ml) in the presence and absence of rhTNF (1 U/ml), WRC-0474[SHA 211] (10 nM) and a corticosteroid (100 nM) in a tissue culture well which had been coated overnight with human fibrinogen. SOD (200 U/ml) was added to matched samples. The supernatants were iced and centrifuged (2000 g×10 minutes) to remove any remaining suspended cells, and the optical density of the supernatants were read at 550 nM against the matched SOD samples, and the nmoles of SOD-inhibitable superoxide released in 90 minutes were calculated.

Effect of WRC-0474[SHA 211] with and without a corticosteroid on TNF-Stimulated PMN Adherence to a Fibrinogen-Coated Surface: Cronstein et al., J. Immunol. 148, 2201 (1992) reported that adenosine binding to A₁ receptors increases PMN adherence to endothelium and matrix proteins and binding to A₂ receptors decreases adherence to these surfaces when the PMN are stimulated with fMLP. Despite this, others have failed to see much of an effect of adenosine (10 μM) on TNF-a-stimulated PMN adherence to matrix proteins. In contrast, adenosine dramatically decreases the oxidative burst of TNF-α-stimulated PMN adhering to matrix proteins (DeLa Harpe, J. J. Immunol., 143 596 (1989)). The experiments described above establish that WRC-0474[SHA 211] decreases TNF-stimulated oxidative activity of PMN adhering to fibrinogen, especially when combined with a corticosteroid.

PMN adherence to fibrinogen was measured as follows as adapted from Hanlon, J. Leukocyte Biol., 50, 43 (1991). Twenty-four well flat-bottomed tissue culture plates were incubated (37° C.) overnight with 0.5 ml of fibrinogen (5 mg/ml) dissolved in 1.5% NaHCO₃. The plates were emptied and each well washed 2× with 1 ml of normal saline. The wells were then filled with 1 ml of HBSS-0.1% human serum albumin containing PMN (1×10⁶/ml) with and without rhTNF-α (1 U/ml), adenosine deaminase (ADA) (1 U/ml), WRC-0474[SHA 211] (10 nM), CGS21680 (30 nM), adenosine (100 nM) and a corticosteroid. The plates were incubated for 90 minutes at 37° C. in 5% CO₂. Following incubation the tissue culture wells were washed free of non-adherent cells with normal saline. The adherent monolayer of PMN was lysed with 0.1% Triton-X, the amount of lactic dehydrogenase (LDH) released from the monolayer assayed (LDH kit, Sigma Co., St. Louis, Mo.), and compared to a standard curve relating the LDH content to PMN numbers.

Additional effects of adenosine A_{2A} agonists on adherent human neutrophil oxidative activity. The bioactivity of test compounds WRC-0474[SHA 211], WRC-0470, WRC-0090 and WRC-0018 were evaluated according to the following method modified from Sullivan, G. W. et al., Int. J. Immunopharmacol., 17, 793-803 (1995). Neutrophils (1×10⁶/ml from Ficoll-Hypaque separation were incubated for 90 minutes in 1 ml of Hanks balanced salt solution containing 0.1% human serum albumin, cytochrome c (120 μM) and catalase (0.062 mg/ml) in the presence and absence of rhTNF-α (1 U/ml), WRC-0474[SHA 211], WRC-0470, WRC-0090 and WRC-0018 (3-300 nM), and a corticosteroid in a tissue culture well which had been coated overnight with human fibrinogen. The supernatants were iced and centrifuged (200 g×10 min) to remove any remaining suspended cells, and the optical densities of the supernatants were read at 550 nM against matched superoxide dismutase (SOD) (200 U/ml) samples. The nmoles of SOD=inhabitable superoxide released in 90 min were calculated.

Application of A_{2A} adenosine receptors with or without an anti-inflammatory compound on balloon angioplasty and gene therapy. Balloon angioplasty is commonly used to treat coronary artery stenosis. Restenosis following balloon angioplasty (BA) occurs in up to 40% of coronary interventions. Holmes et al., American Journal of Cardiology, 53, 77C-81C (1984). Restenosis results from a complex interaction of biologic processes, including (I) formation of platelet-rich thrombus; (ii) release of vasoactive and mitogenic factors causing migration and proliferation of smooth muscle cells (SMC); (iii) macrophage and other inflammatory cell accumulation and foam cell (FC) formation; (iv) production of extracellular matrix; and (v) geometric remodeling. Recently the use of coronary stents and pharmacologic intervention using a chimeric antibody to block the integrin on platelets have been partially successful in limiting restenosis after percutaneous coronary interventions in man. Topol et al., Lencet, 343, 881-886 (1994). Since inflammatory cell infiltration might be central to the response to injury, and restenotic process, and adenosine, activating via A_{2A} adenosine receptors, inhibits tissues inflammatory cell accumulation, we hypothesize that agonists of A_{2A} adenosine receptors+an anti-inflammatory compound will reduce the incidence of restenosis following balloon angioplasty.

In addition, recent advances in local delivery catheters and gene delivery techniques raise the interesting and exciting possibility of administering genes locally into the vessel wall. Nabel et al., Science, 249, 1285-1288 (1990); Leclerc et al., Journal of Clinical Investigation, 90, 936-944 (1992). Adenoviral-mediated gene transfer affords several advantages over other techniques. However, gene expression is only transient, and has been observed for 7-14 days with diminution or loss of expression by 28 days. Lack of persistence may result from host immune cytolytic responses directed against infected cells. The inflammatory response generated by the present generation of adenovirus results in neointimal lesion formation and may thus offset the benefit of a therapeutic gene. Newman et al., Journal of Clinical Investigation, 96, 2955-2965 (1995). An A_{2A} adenosine receptor agonist+an anti-inflammatory compound in combination with adenovirus may improve the efficiency of gene transfer.

Human Neutrophil Preparation:

A one-step Ficoll-Hypaque separation procedure (Ferrante and Thong, 1980) was used to purify human neutrophils from normal heparinized (10 Units/ml) venous blood yielding approximately 98% neutrophils; having about >95% viable as determined with trypan blue containing <50 pg/ml of endotoxin. Following separation, the neutrophils were washed with Hank's balanced salt solution (HBSS) three times.

Neutrophil oxidative activity (luminol-enhanced chemiluminescence): The activated neutrophils emit light from unstable high-energy oxygen species produced by the plasma membrane associated NADPH oxidase and metabolized by cytoplasmic and granule enzymes. The light signal from activated neutrophils can be enhanced by the addition of luminol to the samples. The luminol-enhanced emission of light is stimulated by singlet oxygen, a reactive oxygen species, dependent on both the production of superoxide and mobilization of myeloperoxidase from primary granules (DeChatelet et al., 1982).

The purified neutrophils (1×10⁶/ml) were incubated in HBSS containing 0.1% HSA (1 ml), plus adenosine deaminase (1 Units/ml), +−JR2171 or +−CGS21680, and with TNF-alpha (10 U/ml) for 30 min at 37° C. in a shaking water bath. Then luminol (1×10⁴ M) enhanced f-met-leu-phe (1 μM)-stimulated chemiluminescence was read with a Chronolog Photometer (Chronolog Corp., Havertown, Pa.) at 37° C. for 8 min. Chemiluminescence is reported as relative peak light emitted (=height of the curve) compared to TNFalpha-primed fMLP-stimulated control samples.

Use of Adenosine A_{2A} Agonists to Prevent Ischemic Injury in the Rabbit Spinal Cord. The spinal cord is sensitive to brief periods of ischemia. When the thoracic aorta is clamped, e.g., during surgery, neurologic impairment can result from a lack of blood flow. A rabbit model for spinal cord ischemia resulting in paraplegia is described below.

A laparotomy is performed on a group of rabbits following general anesthesia. The infrarenal aorta is clamped for about forty-five minutes. Cross-clamping the infrarenal aorta causes spinal cord ischemia. The animals are split into two groups. The first group are administered an A_{2A} adenosine agonist, e.g., WRC-0470, 10 μg/kg, infused over about 3 hours. The infusion is started after about 30 minutes of ischemic time. The second group is not administered the A_{2A} adenosine agonist. The animals are allowed to recover for about 48 hours, and assessed for neurologic impairment using the Tarlov (0-5) scoring system. The animals treated with the A_{2A} agonists of formula (I) exhibit substantially less neurological impairment (0-3) than control (untreated) animals.

All patents, patent applications, books and literature cited in the specification are hereby incorporated by reference in their entirety. In the case of any inconsistencies, the present disclosure, including any definitions therein will prevail. The invention has been described with reference to various specific and preferred embodiments and techniques. However, it should be understood that many variations and modifications may be made while remaining within the spirit and scope of the invention. It is therefore to be understood that, within the scope of the appended claims, the invention may be practiced otherwise than as specifically described herein.

What is claimed:

1. A method of treating an inflammatory disorder, comprising administering to a subject suffering from the disorder, a composition comprising:
a) an effective amount of at least one adenosine A_{2A} receptor agonist and
b) an effective amount of at least one anti-inflammatory compound, wherein the anti-inflammatory compound is other than a PDE4 inhibitor, an anti-cholinergic agent, or an anadrenergic β2 receptor agonist.

2. A method of treating an inflammatory disorder, comprising administering to a subject suffering from the disorder, a composition comprising:
a) an effective amount of at least one adenosine A_{2A} receptor agonist and
b) an effective amount of at least one anti-inflammatory compound, wherein the anti-inflammatory compound is other than a PDE4 inhibitor and the adenosine A_{2A} receptor agonist is of formula (I): 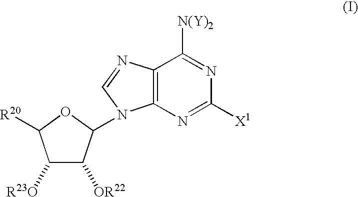
wherein:
X¹ is selected from —OR¹, —NR²R³, —C≡C—Z, and —NH—N═R⁴;
R¹ is selected from:
(a) C₁₋₄ alkyl;
(b) C₁₋₄ alkyl substituted with one or more C₁₋₄ alkoxy, halogen, hydroxy, amino, mono(C₁₋₄ alkyl)amino, di(C₁₋₄ alkyl)amino, and C₆₋₁₀ aryl, wherein aryl may be substituted with one or more halogen, C₁₋₄ alkyl, hydroxy, amino, mono(C₁₋₄ alkyl)amino, di(C₁₋₄ alkyl)amino, and R¹⁵OOC—C₁₋₄ alkyl-;
(c) C₆₋₁₀ aryl; and
(d) C₆₋₁₀ aryl substituted with one or more halogen, hydroxy, amino, mono(C₁₋₄ alkyl)amino, di(C₁₋₄ alkyl)amino, and C₁₋₄ alkyl;
one of R² and R³ has the same meaning as R¹ and the other is hydrogen;
R⁴ is a group having the formula: 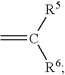
R⁵ and R⁶ are independently selected from H, C₃₋₇-cycloalkyl, or any of the meanings of R¹, provided that R⁵ and R⁶ are not both hydrogen;
Z is selected from (a)-(e):
a) phenyl or naphthyl optionally substituted with one to three groups selected from halogen, C₁₋₆ alkyl, C₁₋₆ haloalkyl, C₁₋₆ alkoxy, C₁₋₆ haloalkoxy, C₂₋₆ alkoxycarbonyl, C₂₋₆ alkoxyalkyl, C₁₋₆ alkylthio, thio, CHO, cyanomethyl, nitro, cyano, hydroxy, carboxy, C₂₋₆ acyl, amino, C₁₋₃ monoalkylamino, C₂₋₆ dialkylamino, methylenedioxy; and aminocarbonyl;
b) a group of formula —(CH₂)ₘ-Het, wherein m is 0 or an integer from 1 to 3 and Het is 5-6 membered heterocyclic aromatic or non-aromatic ring, optionally benzo condensed, containing 1-3 heteroatoms selected from nonperoxide oxygen, nitrogen, and sulfur, linked through a carbon atom or through a nitrogen atom;
c) C₂-C₄ alkenyl or C₃-C₇ cycloalkyl optionally containing unsaturation;
d) 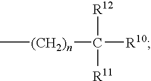
and,
e) C₁-C₁₆ alkyl, optionally comprising 1-2 double bonds, O, S or NY;
R¹⁰ is selected from H, methyl, and phenyl;
R¹² is selected from H, C₁-C₆ alkyl, C₅-C₆ cycloalkyl, C₃-C₇ cycloalkenyl, and phenyl-C₁₋₂ alkyl-;
alternatively, R¹⁰ and R¹², taken together, form a 5- or 6-membered carbocyclic ring;
alternatively, R³ is hydrogen and R² and R⁴, taken together, form an oxo group or a corresponding acetalic derivative;
R¹¹ is selected from OH, NH₂ dialkylamino, halogen, and cyano;
n is selected from 0, 1, 2, 3, and;
Y is individually selected from H, C₁₋₆ alkyl, C₃₋₇ cycloalkyl, phenyl, and phenyl-C₁₋₃ alkyl-;
R²⁰ is selected from —C(═O)NR¹⁶R¹⁷, —COOR¹⁵, and —CH₂OR¹⁵;
each of R¹⁶ and R¹⁷ is independently selected from:
(a) H;
(b) C₃₋₇ cycloalkyl;
(c) C₁₋₄ alkyl;
(d) C₁₋₄ alkyl substituted with one or more C₁₋₄ alkoxy, halogen, hydroxy, —COOR²¹, amino, mono(C₁₋₄ alkyl)amino, di(C₁₋₄ alkyl)amino or C₆₋₁₀ aryl, wherein aryl is optionally substituted with one or more groups selected from halogen, C₁₋₄ alkyl, hydroxy, amino, mono(C₁₋₄ alkyl)amino, di(C₁₋₄ alkyl)amino, and R¹⁵OOC—C₁₋₄ alkyl-;
(e) C₆₋₁₀ aryl; and,
(f) C₆₋₁₀ aryl substituted with one or more halogen, hydroxy, amino, mono(C₁₋₄ alkyl)amino, di(C₁₋₄ alkyl)amino, and C₁₋₄ alkyl;
R²² and R²³ independently represent H, C₁₋₆ alkanoyl, C₁₋₆ alkoxy-C₁₋₆ alkanoyl-, aroyl, carbamoyl, and mono- or di-C₁₋₆ alkylcarbamoyl;
R¹⁵ is selected from H, C₁₋₄ alkyl, C₆₋₁₀ aryl, and C₆₋₁₀ aryl-C₁₋₄ alkyl-; and
R²¹ is independently selected from hydrogen C₁₋₄ alkyl, C₆₋₁₀ aryl, and C₆₋₁₀ aryl-C₁₋₄ alkyl-;
or a pharmaceutically acceptable salt thereof.

3. The method according to claim 2, wherein both R²² and R²³ represent hydrogen.

4. The method according to claim 3, wherein R³ is C₁₋₄ alkyl substituted with C₆₋₁₀ aryl, wherein aryl is substituted with R¹⁵OOC—C₁₋₄ alkyl-.

5. The method according to claim 4, wherein one of R¹⁶ and R¹⁷ is C₁₋₄-alkyl substituted with one or more groups selected from C₁₋₄ alkoxy, halogen, hydroxy, amino, mono(C₁₋₄ alkyl)amino, di(C₁₋₄ alkyl)amino, and C₆₋₁₀ aryl, wherein aryl is optionally substituted with one or more groups selected from halogen, hydroxy, amino, C₁₋₄ alkyl, mono(C₁₋₄ alkyl)amino or di(C₁₋₄ alkyl)amino, and R¹⁵OOC—C₁₋₄ alkyl-.

6. The method according to claim 5, wherein one of R¹⁶ and R¹⁷ is C₆₋₁₀ aryl substituted with one or more groups selected from halogen, hydroxy, amino, mono(C₁₋₄ alkyl)amino, di(C₁₋₄ alkyl)amino, and C₁₋₄ alkyl.

7. The method according to claim 3, wherein R¹⁶ is H and R¹⁷ is selected from C₁₋₄ alkyl, cyclopropyl, and hydroxy-C₂₋₄ alkyl.

8. The method according to claim 3, wherein R²⁰ is ethylaminocarbonyl.

9. The method according to claim 8, wherein X¹ is —NR²R³.

10. The method according to claim 9, wherein R² is H and R³ is selected from C₁₋₄ alkyl substituted with C₆₋₁₀ aryl substituted with R¹⁵OOC—C₁₋₄ alkyl-.

11. The method according to claim 10, wherein R¹⁵ is selected from H, methyl, ethyl, n-propyl, isopropyl, and tert-butyl.

12. The method according to claim 11, wherein R¹⁵ is selected from H, methyl, and ethyl.

13. The method according to claim 12, wherein R¹⁵ is methyl.

14. The method according to claim 12, wherein R¹⁵ is H.

15. The method according to claim 2, wherein the adenosine A_{2A} receptor agonist is selected from a group consisting of:

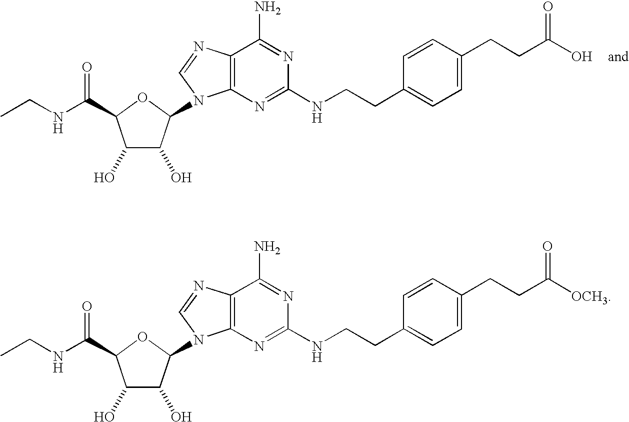

16. The method according to claim 8, wherein X¹ is —C≡C—Z.

17. The method according to claim 2, wherein the adenosine A_{2A} receptor agonist is selected from a group consisting of:

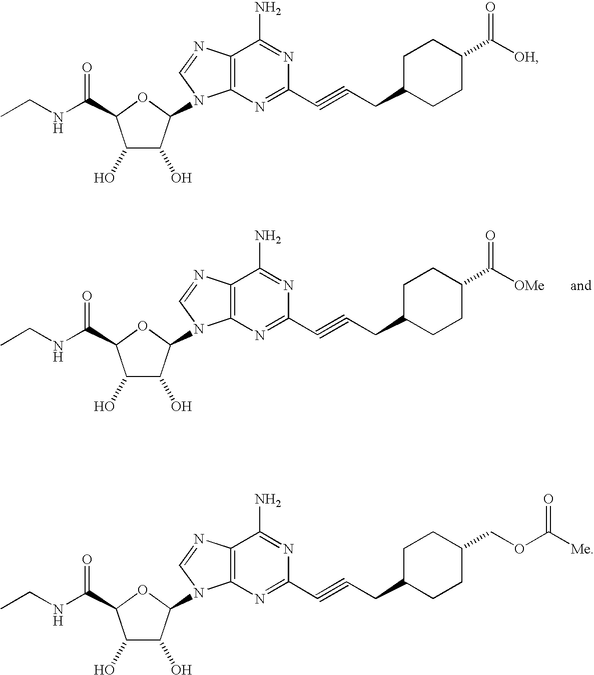

18. The method according to claim 2, wherein R²⁰ is —CH₂OR¹⁵.

19. The method according to claim 18, wherein R¹⁵ is hydrogen.

20. The method according to claim 19, wherein X¹ is —NH—N═R⁴.

21. The method according to claim 1, wherein the adenosine A_{2A} receptor agonist is selected from a group consisting of:

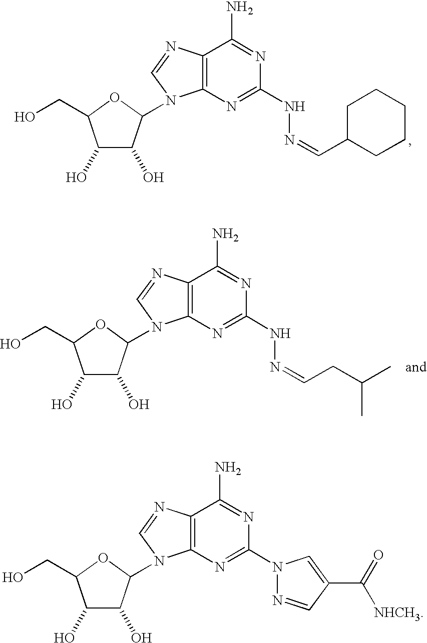

22. The method according to claim 19, wherein X¹ is —OR¹.

23. The method according to claim 2, wherein the adenosine A_{2A} receptor agonist is selected from a group consisting of:

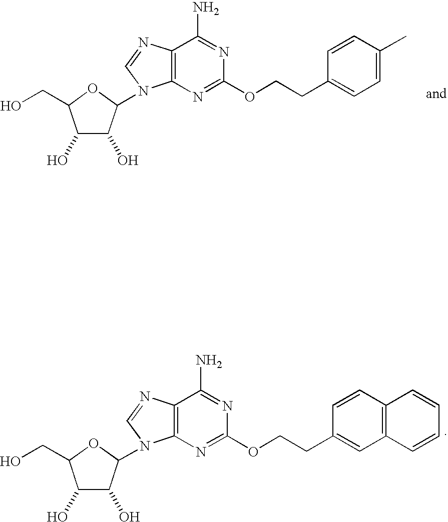

24. The method according to claim 1, wherein the anti-inflammatory compound is selected from the group consisting of the following:
(a) Leukotriene biosynthesis inhibitors, 5-lipoxygenase (5-LO) inhibitors, and 5-lipoxygenase activating protein (FLAP) antagonists;
(b) Receptor antagonists for leukotrienes LTB₄, LTC₄, LTD₄, and LTE₄;
(c) 5-Lipoxygenase (5-LO) inhibitors and 5-lipoxygenase activating protein (FLAP) antagonists;
(d) Dual inhibitors of 5-lipoxygenase (5-LO) and antagonists of platelet activating factor (PAF);
(e) Leukotriene antagonists (LTRAs) of LTB₄, LTC₄, LTD₄, and LTE₄;
(f) Antihistaminic H₁ receptor antagonists;
(g) Gastroprotective H₂ receptor antagonists;
(h) α₁- and α₂-adrenoceptor agonist vasoconstrictor sympathomimetic agents administered orally or topically for decongestant use;
(i) one or more α₁- and α₂-adrenoceptor agonists as recited in (h) above in combination with one or more inhibitors of 5-lipoxygenase (5-LO) as recited in (a) above;
(j) Theophylline and aminophylline;
(k) Sodium cromoglycate;
(l) Muscarinic receptor (M1, M2, and M3) antagonists;
(m) COX-1 inhibitors (NTHEs); and nitric oxide NTHEs;
(n) COX-2 selective inhibitors;
(o) COX-3 inhibitor;
(p) insulin-like growth factor type I (IGF-1) mimetics;
(q) Ciclesonide;
(r) Corticosteroids;
(s) Tryptase inhibitors;
(t) Platelet activating factor (PAF) antagonists;
(u) Monoclonal antibodies active against endogenous inflammatory entities;
(v) IPL 576;
(w) Anti-tumor necrosis factor (TNF-α) agents;
(x) DMARDs;
(y) Elastase inhibitors;
(z) TCR peptides;
(aa) Interleukin converting enzyme (ICE) inhibitors;
(bb) IMPDH inhibitors;
(cc) Adhesion molecule inhibitors including VLA-4 antagonists;
(dd) Cathepsins;
(ee) Mitogen activated protein kinase (MAPK) inhibitors;
(ff) Mitogen activated protein kinase kinase (MAPKK) inhibitors;
(gg) Glucose-6 phosphate dehydrogenase inhibitors;
(hh) Kinin-B₁- and B₂-receptor antagonists;
(ii) Gold in the form of an aurothio group in combination with hydrophilic groups;
(jj) Immunosuppressive agents;
(kk) Anti-gout agents;
(ll) Xanthine oxidase inhibitors;
(mm) Uricosuric agents;
(nn) Antineoplastic agents that are antimitotic drugs;
(oo) Growth hormone secretagogues;
(pp) Inhibitors of matrix metalloproteinases (MMPs);
(qq) Transforming growth factor (TGFβ);
(rr) Platelet-derived growth factor (PDGF);
(ss) Fibroblast growth factor;
(tt) Granulocyte macrophage colony stimulating factor (GM-CSF);
(uu) Capsaicin; and
(vv) Tachykinin NK₁ and NK₃ receptor antagonists.

25. The method according to claim 24, wherein:
(a) the Leukotriene biosynthesis inhibitors, 5-lipoxygenase (5-LO) inhibitors, and 5-lipoxygenase activating protein (FLAP) antagonists are selected from the group consisting of zileuton; ABT-761; fenleuton; tepoxalin; Abbott-79175; Abbott-85761; N-(5-substituted)-thiophene-2-alkylsulfonamides; 2,6-di-tert-butylphenol hydrazones; Zeneca ZD-2138; SB-210661; pyridinyl-substituted 2-cyanonaphthalene compound L-739,010; 2-cyanoquinoline compound L-746,530; indole and quinoline compounds MK-591, MK-886, and BAY x 1005;
(b) the receptor antagonists for leukotrienes LTB₄, LTC₄, LTD₄, and LTE₄ antagonists are selected from the group consisting of phenothiazin-3-one compound L-651,392; amidino compound CGS-25019c; benzoxazolamine compound ontazolast; benzenecarboximidamide compound BIIL 284/260; compounds zafirlukast, ablukast, montelukast, pranlukast, verlukast (MK-679), RG-12525, Ro-245913, iralukast
(CGP 45715A), and BAY x 7195;
(f) the antihistaminic H₁ receptor antagonists antagonists are selected from the group consisting of cetirizine, loratadine, desloratadine, fexofenadine, astemizole, azelastine, and chlorpheniramine;
(h) α₁- and α₂-adrenoceptor agonist vasoconstrictor sympathomimetic agents are selected from the group consisting of propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride;
(n) the COX-2 selective inhibitor are selected from the group consisting of rofecoxib and celecoxib;
(o) the COX-3 inhibitor is acetaminophen;
(r) the Corticosteroids are selected from the group consisting of prednisone, methylprednisone, triamcinolone, beclomethasone, fluticasone, budesonide, hydrocortisone, dexamethasone, mometasone furoate, azmacort, betamethasone, beclovent, prelone, prednisolone, flunisolide, triamcinolone acetonide, beclomethasone dipropionate, fluticasone propionate, mometasone furoate, solumedrol and salmeterol;
(w) the anti-tumor necrosis factor (TNF-α) agents selected from the group consisting of etanercept, infliximab, and D2E7;
(x) the DMARDs is leflunomide;
(y) the Elastase inhibitors are selected from the group consisting of UT-77 and ZD-0892;
(jj) the Immunosuppressive agents selected from the group consisting of cyclosporine, azathioprine, tacrolimus, and methotrexate;
(kk) the anti-gout agents is colchicine;
(ll) the Xanthine oxidase inhibitor is allopurinol;
(mm) the Uricosuric agents are selected from the group consisting of probenecid, sulfinpyrazone, and benzbromarone;
(nn) the antineoplastic agents are selected from the group consisting of vinblastine, vincristine, cyclophosphamide, and hydroxyurea;
(pp) the inhibitors of matrix metalloproteinases (MMPs) are selected from the group consisting of stromelysins, the collagenases, the gelatinases, aggrecanase, collagenase-1 (MMP-1), collagenase-2 (MMP-8), collagenase-3 (MMP-13), stromelysin-1 (MMP-3), stromelysin-2 (MMP-10), and stromelysin-3 (MMP-11); and,
(vv) the Tachykinin NK₁ and NK₃ receptor antagonists are selected from the group consisting of NKP-608C; SB-233412 (talnetant); and D-4418.

26. A pharmaceutical composition comprising at least one adenosine A_{2A} receptor agonist in combination with an anti-inflammatory compound other than a PDE4 inhibitor, an anti-cholinergic agent, and an anadrenergic β2 receptor agonist, each in an amount effective to inhibit the inflammatory disorder.

27. The composition according to claim 26, which is adapted for topical administration.

28. The composition according to claim 26, which is adapted for aerosol administration.

29. The method according to claim 1, wherein the A_{2A} agonist is administered topically.

30. The method according to claim 1, wherein the A_{2A} agonist is administered as an aerosol.
